(19) United States
(12) Patent Application Publication
(10) Pub. No.: US 2008/0142009 A1
(43) Pub. Date: Jun. 19, 2008

(54) Inhalation device

(76) Inventors: Silvio Carrico, Singen (DE); Juergen Greiner-Perth, Gottmadingen (DE); Rene Bommer, Allensbach-Freudental (DE)

Correspondence Address: FLYNN THIEL BOUTELL & TANIS, P.C., 2026 RAMBLING ROAD, KALAMAZOO, MI, 49008-1631, US

(21) Appl. No.: 11/906,066

(22) Filed: Sep. 28, 2007

(30) Foreign Application Priority Data
Sep. 28, 2006 (DE) 10 2006 047 667.0

(51) Int. Cl. A61M 15/00

(52) U.S. Cl. 128/203.15

(57) ABSTRACT

An inhalation device for discharging a pulverulent medium includes a media store that is provided for holding the medium and an outlet channel with an inner wall that is substantially cylindrical at least in a subsection, the outlet channel being designed as a transport path for the medium from the media store into a respiration path opening of a user, and there being provided in the substantially cylindrical subsection of the outlet channel an exit control arrangement that is designed as a baffle and/or exit direction sensor.

### FIELD OF APPLICATION AND PRIOR ART

The invention relates to an inhalation device for discharging a pulverulent medium, having at least one media store that is provided for holding the medium, and an outlet channel with a substantially cylindrical subsection, the outlet channel being designed as transport path for the medium from the media store into a respiration path opening of a user.

Such an inhalation device is disclosed, for example, in EP 0957962 B1. The device described and illustrated there has two trough-shaped media stores that are respectively covered with a protective foil before use such that the medium cannot escape. In order to activate the inhalation device, an outlet channel is pushed in the axial direction into the media store and penetrates the protective foil in the process. In the course of inhalation of the medium, this outlet channel is subsequently used to convey the medium from the media store into a nasal cavity of a user by inhalation.

It is considered to be disadvantageous in this prior art that the pulverulent medium can inadvertently emerge, in particular at the instant of opening of the protective film, and that during inhalation the pulverulent medium cannot be directed sufficiently well in the desired direction owing to the relatively large diameter of the outlet channel.

### OBJECT AND ACHIEVEMENT

The object of the invention consists in providing a generic inhalation device in which the exit of the pulverulent medium is improved with reference to the exit direction and to the avoidance of premature exit.

This object is achieved according to the invention by means of an inhalation device of the type mentioned at the beginning in the case of which there is arranged in the substantially cylindrical subsection of the outlet channel an exit control arrangement with at least one control element, which arrangement is designed as baffle and/or exit direction sensor.

Such an inhalation device has a preferably multipartite housing inside which the media store is arranged and on which the outlet channel is provided such that is accessible from outside and permits inhalation of the pulverulent medium located in the media store. In this case, inhalation is performed via the mouth or the nose. In a preferred embodiment, the media store is sealed before activation such that no medium can exit. The entire outlet channel preferably has a substantially cylindrical inner wall, outlet channels with inner walls of slightly conical shape also being regarded as cylindrical in the meaning of the invention. Provided in a cylindrical or slightly conical subsection of the outlet channel, preferably in the region of a side of the outlet channel averted from the media store, is the exit control arrangement that consists of control elements that extend in the cross section of the cylindrical subsection. These control elements can be designed, for example, as flat guide surfaces extending in the cross sectional direction, or else as single-sided or circumferential steps or beads on the inner wall. When the exit control arrangement is intended, in particular, to serve as baffle, the control elements are arranged such that even in the event of an opened media store, that is to say directly before the proper use of the inhalation device, an inadvertent shaking of the inhalation device, or else a stirring up of the pulverulent medium by a brief air current in the region of the media store does not lead to exiting of the medium from the inhalation device, since the medium that has reached the outlet channel strikes against the exit control arrangement and remains lying there, at least for the greater part, or falls back into the media store. This is advantageous, in particular, in the case of inhalation devices in which the medium is sealed off from outside by a protective foil in the as-supplied state, and in which this protective foil is penetrated by pushing in the outlet channel. This penetration is preceded by pressing the outlet channel into the protective foil, which leads to an excess pressure in the media store and, at the instant when the outlet channel is pressed, can result in stirring up of the pulverulent medium, something which can cause it to exit from the inhalation device in the event of lack of a baffle. In addition, in the case of multipartite housings stirring up the medium can be favored by the housing parts meeting one another upon activation and coming to bear against one another through impacting, a shock being caused that acts impulsively on the medium. If the exit control arrangement serves as exit direction sensor, it is preferably constructed from control elements that lend a specific direction to the air current with the pulverulent medium in the course of inhalation in the region of an exit opening of the outlet channel without substantially increasing the air resistance. This is preferably performed by means of flat guides whose surface normal encloses an angle of between 0 and 45 degrees in the principal direction of extent of the outlet channel.

In a development of the invention, the exit control arrangement is integrally formed in one piece on the inner wall of the outlet channel.

Such a refinement is particularly cost effective in production, since only slight modifications need to be undertaken to the method of producing the outlet channel. For simple control arrangements, in particular, for example in the case of exit direction sensors that consist merely of a simple guide surface extending into the outlet channel, an integral formation directly onto the inner wall of the outlet channel is obvious.

In an alternative development of the invention, the exit control arrangement is designed as a separate control component that is inserted into the cylindrical subsection.

Such a modular design in which the outlet channel and the exit control component are not designed in one piece, is obvious particularly when, for example, various exit control arrangements are to be provided in identical outlet channels as a function of the medium used. By virtue of the fact that the outlet channels can be used for various exit control arrangements, they can be produced in large numbers of items, and therefore very cost effectively. Instead of adapting the entire cylindrical subsection in a way specific to media and/or application, such an adaptation can be limited to the exit control component. Separating the exit control arrangement and the outlet channel is also advantageous from points of view in production engineering, since it is easier to produce the exit control arrangement as a separate component, particularly in the case of complex exit control arrangements. If appropriate, separate fabrication can also constitute the sole possibility of production in the case of complex exit control arrangements.

The fixing of the exit control component in the outlet channel can be achieved, for example, by means of latching means, such as latching webs and latching grooves, provided for the purpose. A particularly simple form of the fixing is achieved when the outlet channel has a slightly conical inner wall into which the exit control component is pushed in the tapering direction of the cone such that a force-closed connection results between the exit control component and outlet channel. Moreover, alongside force-closed and self-closed connections it is also possible to conceive of bonded connections such as adhesively bonded connections, and to prefer them in the individual case.

In one development of the invention, a projection of the exit control arrangement is closed with reference to a principal axis of extent of the cylindrical subsection of the outlet channel.

Here, the projection referred to a principal axis of extent is understood as a projection onto an imaginary plane whose surface normal is formed by the principal direction of extent. This form of the exit control arrangement means that powder particles of the medium that undesirably pass from the media store into the outlet channel cannot traverse the latter without changing their direction of movement. It follows that without a carrier air current that is required for this purpose but is not present, or is present only at insufficient strength, in the event of a merely inadvertent shaking of the inhalation device, or in the event of short air blasts, for example due to the bursting open of the protective foil of the media store, the medium does not exit. An inhalation device having an exit control arrangement in accordance with this development therefore particularly constitutes a good baffle. A closed projection in the sense of the invention is also understood as a projection in the case of which narrow, linear free areas remain unclosed, since a complete projection without gaps places very high demands on production. By accepting linear free areas, it is possible to apply production methods that are substantially more favorable and of lower wear with reference to tools.

In one development of the invention, the exit control component has a fastening section that is in the shape of a hollow cylinder and bears against the inner wall of the cylindrical subsection of the outlet channel.

The hollow cylindrical fastening section is here at least partially open at both end faces. It is preferably dimensioned such that it bears against the inner wall of the outlet channel in such a way that it forms a force-closed connection therewith. Alternative refinements provide that a clearance fit is present between it and the inner wall of the outlet channel, the fixing being provided by latching means or a bonded connection. The use of an exit control component with a hollow cylindrical fastening section is advantageous, in particular, with reference to handling, in particular mounting.

In one development of the invention, the exit control arrangement has at least two control elements that are provided at various levels with reference to a principal axis of extent of the cylindrical subsection of the outlet channel, and that are preferably designed as guide surfaces.

Various levels are understood in this context as various distances by which the various control elements are spaced apart from the ends of the outlet channel in the principal direction of extent. The provision of various control elements at various levels enables the cross section to be completely closed in the projection, and yet, at the same time, permits a largely unimpeded air current that is required for the proper use for inhalation. It can be expedient in the case of particular fields of application to keep the distance between control elements low in order specifically to create a high air resistance. An increased air resistance is expedient in the case of media that are ideally taken in only small doses or at a low rate. It can be sensible in order to increase the air resistance specifically to provide more than two control elements and to arrange these in particular such that the air current is subjected several times to a change in direction.

In one development of the invention, the at least one control element has a guide surface in the shape of a circular segment or circular sector.

Such circular segment or circular sector guide surfaces are particularly easy to produce in terms of production engineering. A single such guide surface is suitable, in particular, as an exit direction sensor, while a number of such guide surfaces at various levels can be used to implement a baffle of simple design.

In one development of the invention, the at least one guide surface is aligned in the outlet channel in such a way that its surface normal encloses an angle of greater than 0° and less than 90° with a principal direction of extent of the cylindrical subsection.

Such a guide surface designed as an inclined plane constitutes a particularly expedient and simple form of an exit direction sensor. Angles of greater than 45°, in particular, are of advantage for such an application, since they associate an effective deflection of media particles with a comparatively slight increase in the flow resistance. A refinement with a number of guide surfaces that are arranged obliquely in the outlet channel is suitable for preventing a completely closed projection of the exit control arrangement without there being a significant rise in the flow resistance in the course of the proper use of the inhalation device. Here, the guide surfaces are expediently arranged obliquely in the outlet channel in such a way that they extend away, starting from the inner wall of the outlet channel, to the middle of the outlet channel in the direction of an exit opening of the outlet channel.

In one development of the invention, at least one of the control elements is provided at a level inside the cylindrical subsection of the outlet channel up to which the hollow cylindrical fastening section does not extend.

This enables the provision of a baffle that is inserted into the outlet channel in the form of a separate exit control component without leading to a serious increase in the flow resistance. It is thus possible to provide a completely closed projection of the exit control arrangement by virtue of the fact that there is provided at the level of the hollow cylindrical fastening section a first control element that seals a first part of the cross sectional surface of the outlet channel in the projection, while the remaining part of the projection is sealed by a second control element that is arranged below or above the hollow cylindrical fastening section and connected to the latter, preferably by a web extending in the principal direction of extent of the outlet channel. The first control element at the level of the hollow cylindrical fastening section can also be formed by the latter itself.

In one development of the invention, the exit control arrangement, or a part thereof, in particular the control elements designed as guide surfaces, are designed to be elastically deformable.

In the case of such a refinement, control elements are preferably designed with reference to their deformability such that without a force acting on them they constitute a baffle that seals the outlet channel at least in a substantial subsection. If a sufficiently large force acts on the control elements via the air current caused during inhalation, they are completely or partially deformed such that it enlarges the free cross section in the outlet channel. There is a consequent reduction in the flow resistance, and the inhalation operation is facilitated. Particularly expedient in this case is a refinement in the case of which the guide surfaces themselves are not elastically deformable, or are so only to a slight extent, but are connected by webs to the inner wall of the outlet channel or of the hollow cylindrical fastening section that, for their part, are elastically deformable, and thus function in the manner of pivot hinges.

In one development of the invention, the exit control arrangement has at least two control elements, one control element forming an outer taper arranged on the inner wall of the outlet channel or of the fastening section, and the other control element being arranged in the outlet channel in a preferably centered fashion.

In the case of this refinement, a completely closed or virtually completely closed projection is accordingly achieved by means of two control elements, the first control element covering an annular outer region, and the second control element covering an inner region, lying therein, of the projection. During the inhalation operation, the air current in the region of the first control element is guided past through the inner opening formed by the latter, and in the region of the second control element it is guided past the latter on all sides. Such a refinement of the exit control arrangement has the particular advantage that the air current is not pressed into a specific direction inside the outlet channel, but flows through an exit opening of the outlet channel in a homogeneous and centered fashion. Particular preference is given to embodiments in which the control elements have a form that leads to a reduction in the flow resistance. This can be achieved, in particular, by a conical shape in the case of the centered control element, the cone tapering counter to the direction of media discharge. Moreover, embodiments in which the control element arranged on the inner wall is formed directly by the hollow cylindrical fastening section are also considered as advantageous.

In one development of the invention, the at least one control element comprises a helical channel whose principal direction of extent corresponds to the principal direction of extent of the outlet channel.

An exit arrangement having such a helical channel permits a simple implementation of a completely closed projection, as well as of an exit direction sensor. Moreover, in conjunction with a low space requirement the helical channel constitutes a simple possibility for achieving a homogenization of the media/air mixture to be inhaled.

### BRIEF DESCRIPTION OF THE DRAWINGS

Further advantages and features of the invention follow from the claims and the subsequent description of a number of preferred exemplary embodiments of the invention that are illustrated with the aid of the drawings, in which:

FIG. 1 shows an inventive inhalation device having a particularly simple form of an exit control arrangement that is integrally formed directly on the wall of the outlet channel of the inhalation device,

FIGS. 2a to 2d show the inhalation device of FIG. 1 in a number of stages of activation and of use;

FIG. 3 shows a second embodiment of an inventive inhalation device whose exit control arrangement corresponds to the embodiment of FIG. 1 but is implemented as a separate component,

FIGS. 4 to 11 show various variants of the exit control arrangement in respectively different representations, and

FIG. 12 shows a third embodiment of an inventive inhalation device in the case of which an exit control arrangement and a tip for opening a media store are designed as a common, unipartite component.

### DETAILED DESCRIPTION OF THE EXEMPLARY EMBODIMENTS

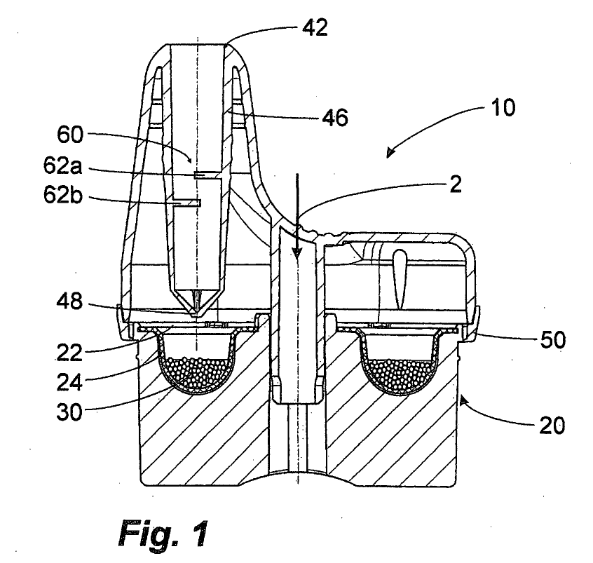

FIG. 1 shows a first embodiment of an inventive inhalation device 10 that is composed of a lower housing part 20 and an upper housing part 40. Provided in the lower housing part 20 are two media stores 24 that respectively contain a specific quantity of a pulverulent medium 30. The trough-shaped media stores 24 are covered by protective foils 22 and sealed with respect to the environment. The upper housing part 40 has an outlet channel 42 whose inner wall 46 tapers conically in the direction of the media store 24.

The upper housing half 40 is designed such that it can be displaced in a joining direction 2 with respect to the lower housing part 20, and this serves the purpose of punching open the protective foil 22 with the aid of a tip 48 of the outlet channel 42 that is integrally formed on an inner wall section 46. In the state illustrated in FIG. 1, a relative displacement is, however, blocked by a removable safety ring 50 that serves as an originality protection and prevents inadvertent activation of the inhalation device 10.

Arranged in the outlet channel 42 inside the inner wall section 46 is an exit control arrangement 60 that consists of two guide surfaces 62a, 62b which are each approximately semicircular and are integrally formed at a different level on opposite sides of the inner wall section 46 of the outlet channel 42.

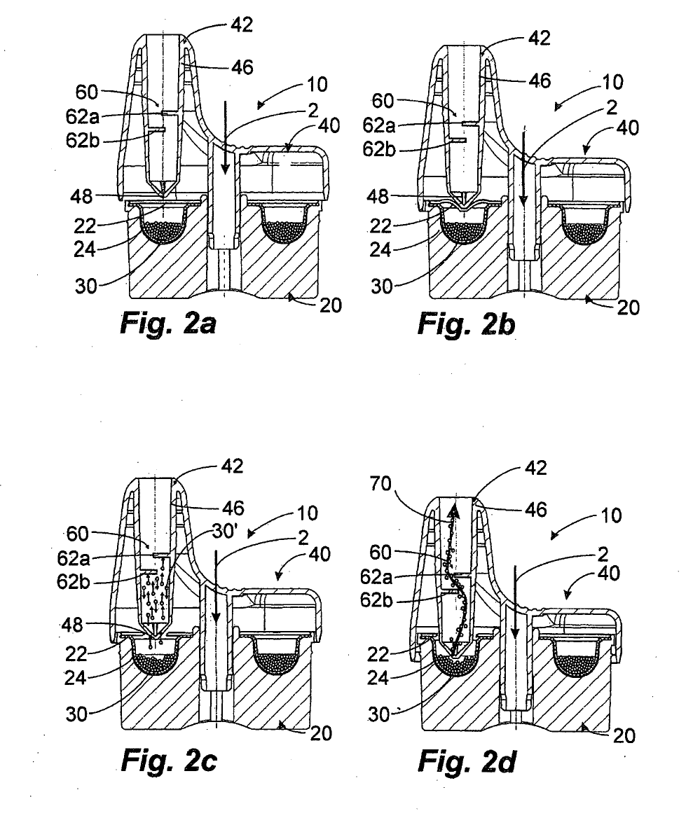

FIGS. 2a to 2d illustrate the mode of operation of the inhalation device 10 and, in particular, of the exit control arrangement 60. Starting from the original state, which is illustrated in FIG. 1, after removal of the safety ring 50—illustrated in FIG. 2a—the two housing parts 20, 40 can be pressed into one another, and the inhalation device can thereby be activated.

As is to be gathered from FIG. 2b, in this case the lower tip 48 of the inner wall section 46 sits against the protective foil 22 of one media store 24. The protective foil 24 is deformed in this case and an excess pressure is produced in the media store 22.

Pressing inward is continued until—as illustrated in FIG. 2c—the protective foil 24 tears. The air thereby escaping from the media store 22 entrains a small portion 30′ of the pulverulent medium 30. This portion 30′ passes into the outlet channel 42. The effect is further amplified by the displacement of the air from the upper housing part 40, since this likewise partially escapes into the outlet channel 42 through the media store 22. The portion 30′ of the pulverulent medium 30 that passes into the outlet channel 42 can, however, not exit outward through the outlet channel 42 because of the exit control arrangement 60. Instead of this, the powder particles 30′ predominantly bounce against the guide surfaces 62a, 62b and fall back into the media store 24.

Only after the outlet channel 42 is laid against a nasal cavity of the user and the latter produces a continuous inhalation air current 70, can the pulverulent medium 30 that is entrained by the air current 70 exit outward from the exit channel 42 past the guide surfaces 62a, 62b.

The illustrated exit control arrangement therefore has the effect that a discharge is possibly only when a continuous air current caused by inspiration conveys the pulverulent medium outward as carrier thereof. An impulsive short air blast such as is caused, for example, by the tearing of the pressurized protective foil 24 is not sufficient to convey the pulverulent medium outward past the exit control arrangement. Protection against exit is likewise ensured in the time period after activation and before use if the inhalation device 10 is inadvertently moved jerkily. The portion of the medium 30 that may pass through inertia into the exit channel 42 is prevented overwhelmingly by far from exiting by the guide surfaces 62a, 62b.

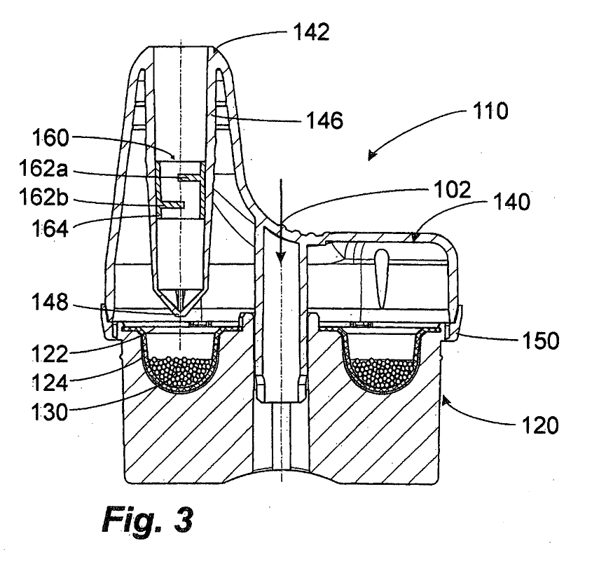

FIG. 3 shows an alternative embodiment that differs from the embodiment of FIG. 1 only in that an exit control arrangement is present as a separate exit control component 160. This exit control component 160 consists of a hollow cylinder 164 that approximately corresponds with reference to its outside diameter to the inside diameter of the inner wall 146 of the outlet channel 142, and of guide surfaces 162a, 162b that correspond to the embodiment of FIG. 1 except for a slightly smaller area of the guide surfaces 62a, 62b. The mode of operation of this second embodiment is identical to the mode of operation of the first embodiment of FIG. 1. Differences arise only with regard to the production and to the particular flow conditions caused by the smaller cross section of the hollow cylinder 164 as against the outlet channel 42. During production, the upper housing part 140 and the exit control component 160 are produced separately and subsequently joined together. This modular design is to be preferred, in particular, whenever a multiplicity of different exit control arrangements are to be produced which are intended as a whole to be used in otherwise identically designed upper housing parts 140, or when the upper housing part 140 is intended to be capable of use both for inhalation devices with and without an exit control component 160.

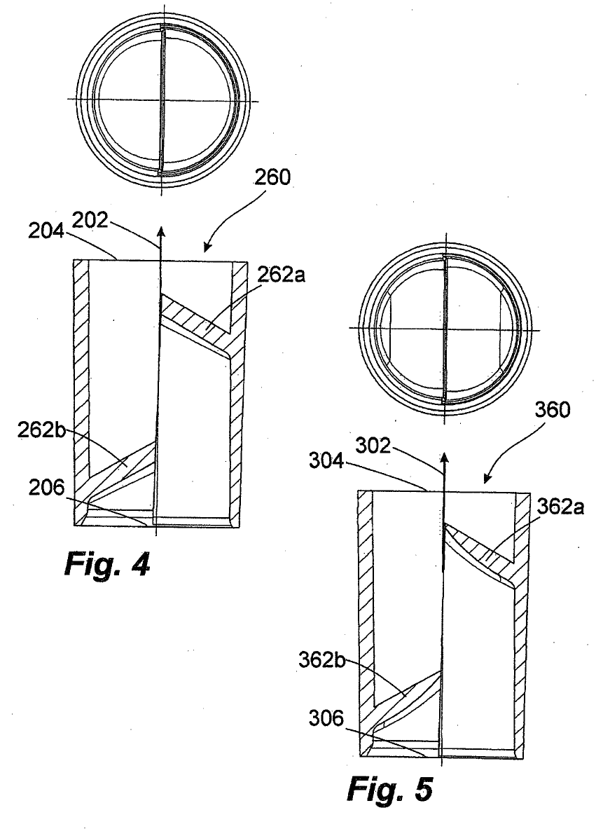

FIGS. 4 to 11 show various advantageous exit control arrangements, these geometries respectively being implemented as separate exit control components. However, it may be pointed out that the exit control arrangements can likewise also be integrally formed in one piece with the outlet channel and on the inner wall thereof.

FIGS. 4 to 6 respectively show exit control components 260, 360, 460 with in each case two control elements 262a, 262b, 362a, 362b, 462a, 462b designed as guide surfaces. The embodiments of FIGS. 4 and 5 differ from the embodiment of FIG. 1 in that the guide surfaces 262a, 262b, 362a, 362b are respectively arranged in the outlet channel such that they rise toward the middle of the outlet channel in the direction of an outlet side 240, 304. This results in a reduction in flow resistance against the embodiment of FIG. 1. This facilitates the inhalation operation without appreciably diminishing the suitability of the exit control components 260, 360 with regard to their function as baffle. The two embodiments of FIGS. 4 and 5 differ from one another in that the guide surfaces 362a, 362b of the exit control component 360 taper toward the middle, and therefore cause a lesser flow resistance.

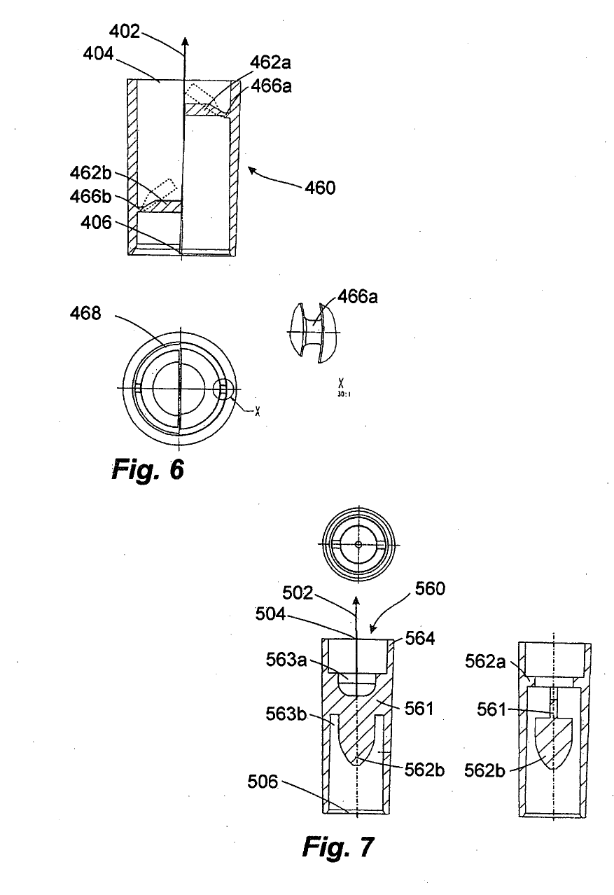

The embodiment of FIG. 6 resembles the abovenamed embodiments to the extent that likewise two approximately semicircular guide surfaces 462a, 462b are provided as control elements. In a departure from the abovenamed embodiments, these guide surfaces 462a, 462b are, however, connected to the hollow cylinder 464 only by means of a narrow web 466a, 466b that is tapered in relation to the thickness of the guide surfaces. A free narrow gap 468 therefore results in a circumferential region 468 in which the guide surfaces 462a, 462b are not connected to the hollow cylinder 464. Because of the narrow width of the gap 468, the effect of the exit control arrangement 460 as baffle is not substantially reduced. However, the joining of the guide surfaces 462a, 462b to the hollow cylinder by means of narrow webs 466a, 466b leads advantageously to the fact that the guide surfaces are deflected in the exit direction 404 in the course of the inhalation operation by the suction action 462a, 462b. The deflected position is illustrated by dashes in FIG. 6. The reduction in air resistance that is attained by the deflection facilitates the inhalation operation for the user.

FIGS. 7 to 10 show exit control arrangements that, in a departure from the preceding exemplary embodiments, exhibit no guide surfaces in the shape of circular segments, but instead form a first circumferential control element arranged at the edge, and a second, centrally arranged control element that jointly likewise form a baffle.

In the case of the embodiment of FIG. 7, the first control element 562a is constructed inside a hollow cylinder 564 as a ring, integrally formed on the inside of the hollow cylinder 564, with approximately square cross section. This first control element 562a surrounds an opening 563a. The second control element 562b is arranged below the first control element 562a in a central fashion inside the hollow cylinder 564 and has a shape that tapers conically in the direction of the entrance end 506. A through gap 563b lies between the second control element 562b, whose diameter corresponds to the diameter of the opening 563a, and the hollow cylinder 564. The second control element 562b is connected to the hollow cylinder 564 via narrow webs 561.

This exit control arrangement of FIG. 7 also has a virtually completely closed projection such that without a continuous air current pulverulent medium entering at the entrance end 506 is prevented from exiting at the exit end 504 by the first control element 563a or the second control element 563b. Given the presence of an air current caused by inhalation, however, the pulverulent medium can pass through the through gap 563b and the through opening 563a from the entrance end 506 to the exit end 504.

As against the embodiments of FIGS. 1 to 6, it is advantageous in this refinement that the exit direction of the pulverulent medium corresponds approximately to the principal direction of extent of the outlet channel owing to the largely rotationally symmetrical arrangement of the control elements. This is advantageous, in particular, in the case of those inhalation devices that need not necessarily be used in a defined position.

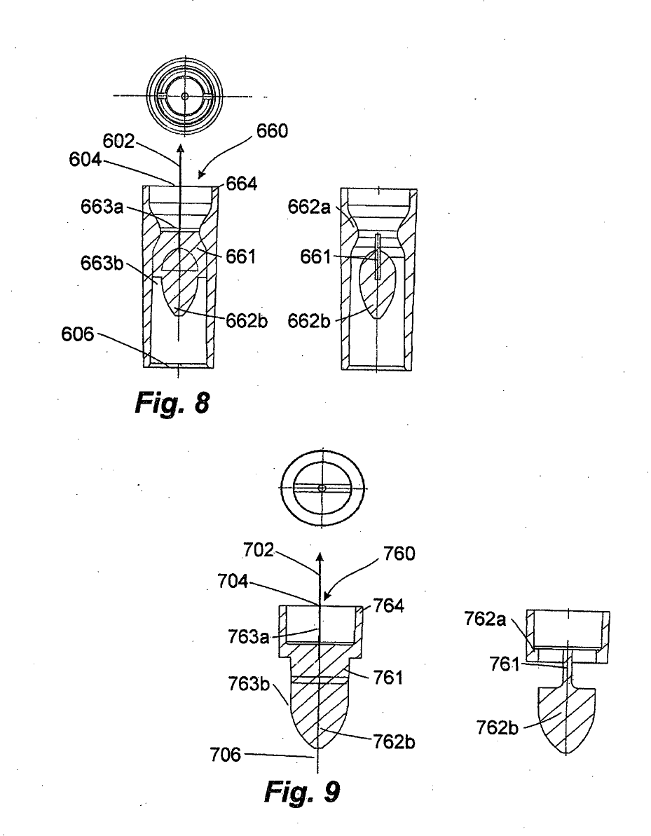

The embodiment of FIG. 8 corresponds largely to the embodiment of FIG. 7. However, in a departure from the latter the first control element 662a is designed as a simple tapering of the hollow cylinder 664. Sharp edges that interfere hydrodynamically with regard to the suction action required for the inhalation operation are not present in this case.

FIG. 9 shows a further embodiment of an exit control component 760. This resembles the exit control component of FIG. 7. However, as a departure therefrom it has a substantially shortened hollow cylinder 764 that does not extend as far as into the region of the second control element 762b. Moreover, the first control element 762b is designed merely as only a slight widening of the wall thickness of the hollow cylinder 764. The advantage of this embodiment is that the diameter of the outlet channel into which the exit control component 760 is inserted is not reduced over the entire length by the hollow cylinder 764 as is the case for the embodiments of FIGS. 7 and 8. The result is improved flow properties with a lesser flow resistance during the inhalation operation.

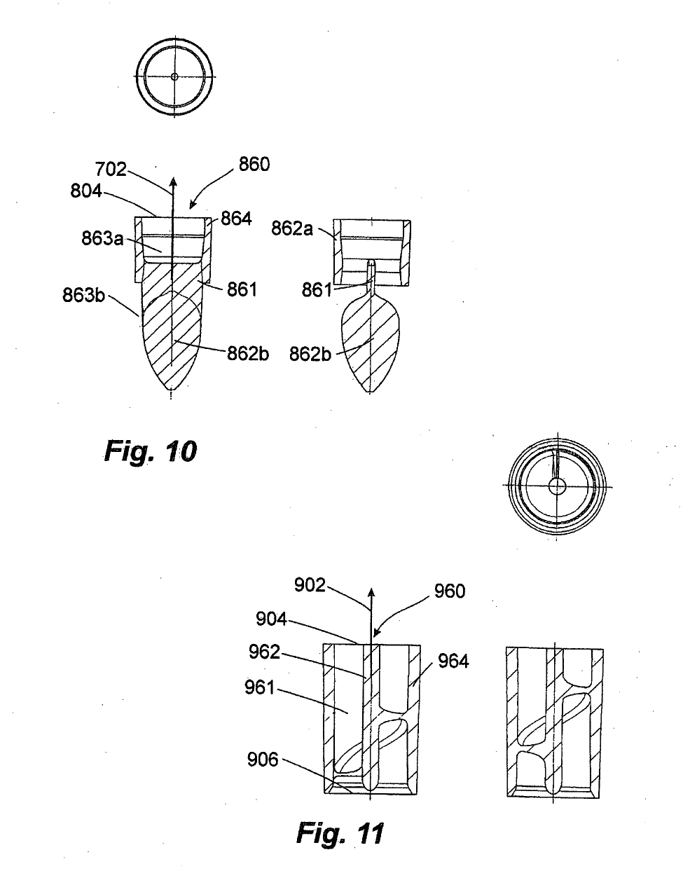

FIG. 10 shows yet a further embodiment, which likewise utilizes this advantageous effect. As against the embodiment of FIG. 9, however, here the hollow cylinder 864 itself directly forms the first control element 862a.

FIG. 11 shows a further embodiment of an exit control arrangement differing substantially therefrom. In this case, a structure with a helical through channel 961 is constructed as control element 962 inside the hollow cylinder 960. This control element 962 likewise constitutes a closed projection and is therefore likewise suitable as baffle. Moreover, such an embodiment offers the advantage that inside the through channel 961 the air/media mixture to be inhaled will be stirred up and thus advantageously homogenized.

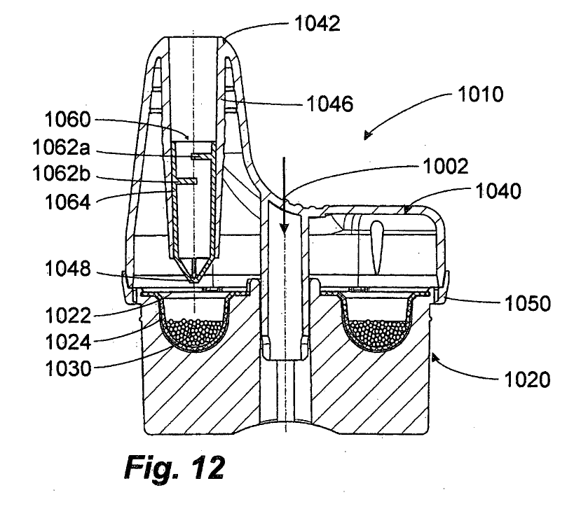

The embodiment of FIG. 12 resembles the embodiment of FIG. 3. In a departure, the tip 1048 is, however, not integrally formed on the cylindrical section 1046 of the outlet channel 1042, but forms together with the exit control arrangement 1060 a unipartite component that is fastened in a force-closed fashion in the outlet channel 1042. Such a configuration increases the flexibility with regard to the configuration of the outlet channel and of the exit control arrangement.

1. Inhalation device (10) for discharging a pulverulent medium (30), having
at least one media store (24) that is provided for holding the medium (30), and
an outlet channel (42) with a substantially cylindrical subsection (46),
the outlet channel (42) being designed as transport path for the medium from the media store (30) into a respiration path opening of the user,
characterized in that
there is arranged in the substantially cylindrical subsection (46) of the outlet channel (42) an exit control arrangement (60; 160; 260; 360; 460; 560; 660; 760; 860; 960) with at least one control element (62a, 62b; 162a, 162b; 262a, 262b; 362a, 362b; 462a, 462b; 562a, 562b; 662a, 662b; 762a, 762b; 862a, 862b; 962), which arrangement is designed as baffle and/or exit direction sensor.

2. Inhalation device according to claim 1,
characterized in that
the exit control arrangement (60) is integrally formed in one piece on an inner wall of the outlet channel (42).

3. Inhalation device according to claim 1,
characterized in that
the exit control arrangement (160; 260; 360; 460; 560; 660; 760; 860; 960) is designed as a separate exit control component (160; 260; 360; 460; 560; 660; 760; 860; 960) that is inserted into the cylindrical subsection (146).

4. Inhalation device according to Claim 1,
characterized in that
a projection of the exit control arrangement (60; 160; 260; 360; 460; 560; 660; 760; 860; 960) is closed with reference to a principal axis of extent (2; 102; 202; 302; 402; 502; 602; 702; 802; 902) of the cylindrical subsection (46; 146) of the outlet channel (42; 142).

5. Inhalation device according to claim 3,
characterized in that t
he exit control component (160; 260; 360; 460; 560; 660; 760; 860; 960) has a fastening section (164; 264; 364; 464; 564; 664; 764; 864; 964) that is in the shape of a hollow cylinder and bears against the inner wall of the cylindrical subsection (146) of the outlet channel (142).

6. Inhalation device according to Claim 1,
the exit control arrangement (60; 160; 260; 360; 460; 560; 660; 760; 860) has at least two control elements (62a, 62b; 162a, 162b; 262a, 262b; 362a, 362b; 462a, 462b; 562a, 562b; 662a, 662b; 762a, 762b; 862a, 862b) that are provided at various levels with reference to a principal axis of extent (2; 102; 202; 302; 402; 502; 602; 702; 802; 902) of the cylindrical subsection (46; 146) of the outlet channel (42; 142), and that are preferably designed as guide surfaces (62a, 62b; 162a, 162b; 262a, 262b; 362a, 362b; 462a, 462b).

7. Inhalation device according to Claim 1,
characterized in that
the at least one control element (62a, 62b; 162a, 162b; 262a, 262b; 362a, 362b; 462a, 462b) has a guide surface (62a, 62b; 162a, 162b; 262a, 262b; 362a, 362b; 462a, 462b) in the shape of a circular segment or circular sector.

8. Inhalation device according to claim 7,
characterized in that
the at least one guide surface (262a, 262b; 362a, 362b; 462a, 462b) is aligned in the outlet channel in such a way that its surface normal encloses an angle of greater than 0° and less than 90° with a principal direction of extent (202, 302, 402) of the cylindrical subsection.

9. Inhalation device according to claim 5,
characterized in that
at least one of the control elements (762b; 862b) is provided at a level inside the cylindrical subsection of the outlet channel up to which the hollow cylindrical fastening section (764; 864) does not extend.

10. Inhalation device according to Claim 1,
characterized in that
the exit control arrangement (460) or a part (462a, 462b) thereof, in particular the control elements (462a, 462b) designed as guide surfaces (462a, 462b), are designed to be elastically deformable.

11. Inhalation device according to claim 2,
characterized in that
the exit control arrangement (560; 660; 760; 860) has at least two control elements (562a, 562b; 662a, 662b; 762a, 762b; 862a, 862b), one control element (562a, 662a; 762a; 862a) forming an outer taper arranged on the inner wall of the outlet channel or of the fastening section (564; 664; 764; 864), and the other control element (562b; 662b; 762b; 862) being arranged in the outlet channel in a preferably centered fashion.

12. Inhalation device according to Claim 1,
characterized in that
the control element (962) comprises a helical channel, the principal direction of extent (902) of the helix corresponding to the principal direction of extent of the outlet channel.
